(19) United States
(12) Patent Application Publication
(10) Pub. No.: US 2004/0181150 A1
(43) Pub. Date: Sep. 16, 2004

(54) Methods and apparatus for accessing and treating body lumens

(75) Inventors: Michael A. Evans, Palo Alto, CA (US); Denise M. Demarais, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BACCHUS VASCULAR, INC., Santa Clara, CA

(21) Appl. No.: 10/812,536

(22) Filed: Mar. 29, 2004

Related U.S. Application Data
(63) Continuation of application No. 09/388,294, filed on Sep. 1, 1999.

(51) Int. Cl. A61B 6/00

(52) U.S. Cl. 600/433

(57) ABSTRACT

Access to blood vessels and other body lumens is provided from spaced-apart access penetrations. A penetrating device is introduced through a first access penetration to a target site within the body lumen and then used to penetrate outwardly from the body lumen to an external surface on an organ or the patient's skin. A guidewire may then be deployed through the penetrating device, and the penetrating device removed to leave the guidewire in place. Catheters and other interventional and diagnostic devices may then be introduced to the target site from either or both of the first and second access penetrations over the same guidewire.

### CROSS-REFERENCES TO RELATED APPLICATIONS

The present application is a continuation of U.S. application Ser. No. 09/388,294 (Attorney Docket No. 19744P-000300US), filed Sep. 1, 1999, the full disclosure of which is incorporated herein by reference.

### BACKGROUND OF THE INVENTION

1. Field of the Invention

The present invention relates generally to medical apparatus and methods and more particularly to methods and apparatus for providing access to body lumens between spaced-apart access points on the lumen.

In recent years, a wide variety of intravascular and intraluminal procedures have been developed where catheters and other treatment devices may be introduced to a luminal target site over a guidewire. For vascular procedures, a guidewire is typically introduced to the blood vessel through an access penetration formed either by a surgical cut-down procedure or by using a needle and guidewire exchange procedure commonly referred to as the Seldinger technique. In either case, a single guidewire is advanced from the access site to a target location within the blood vessel or other lumen. Once the guidewire is in place, it acts as a rail or track, permitting various diagnostic and interventional catheters to be introduced to the target location.

While these techniques have proven to be enormously successful and beneficial, the use of a single guidewire which is not anchored at its distal end is limited in certain respects. For example, the inability to place tension on the guidewire can make advancement of a catheter over the guidewire difficult, particularly when very tortuous or narrow stenotic regions need to be crossed. Second, placement of a single guidewire through a single entry point in the blood vessel or other lumen allows access from only a single side of a treatment site. In many circumstances, it would be desirable to diagnose and/or treat a target location in a blood vessel or other body lumen from both sides simultaneously.

In order to provide tension on a guidewire, it has been proposed to form a second percutaneous access site to a patient's vasculature and to pull a previously placed guidewire out through the second access site. For example, it has been proposed to use a second guidewire having a loop or other capture device at its distal end to tether the first guidewire and pull the first guidewire through the second access site. While successful, the need to create a second access location, either by cut-down or the Seldinger technique, is more invasive and inefficient. Moreover, the need to capture the first guidewire with a second guidewire or snare is technically difficult. Thus, present techniques for placing guidewires between spaced-apart access locations in a blood vessel or other body lumen are in need of significant improvement.

It has also been proposed to treat target sites within a patient's vasculature using two catheters introduced from opposite sides of the treatment location. For example, it has been proposed to introduce an interventional catheter, such as an embolectomy catheter, from one side of a treatment location and to introduce an aspiration or a thrombus capture catheter from the other side of the treatment location. By applying aspiration through the aspiration catheter, occlusive material which is dislodged using the embolectomy catheter may be efficiently and completely removed from the vasculature. While theoretically promising, such techniques have been difficult to implement at least in part because of the difficulty in separately positioning the two treatment catheters on opposite sides of the treatment site. In particular, the need to place two separate guidewires and position those guidewires at a single treatment site within a common blood vessel can be quite difficult.

For these reasons, it would be desirable to provide improved methods and apparatus for accessing target sites in blood vessels and other body lumens from opposite sides of the target site. In particular, it would be desirable to provide improved methods and apparatus for forming a second access penetration spaced-apart from a first access penetration in a body lumen, where the first access penetration will typically be formed by conventional techniques. It would be still further desirable to provide catheters and tools which may be introduced through (and optionally create) a first access penetration into a body lumen and thereafter used to form the second access penetration. Moreover, it would be desirable if such access tools and catheters were capable of placing a guidewire between the first and second access sites after the second access site has been formed. Additionally, it would be desirable to provide improved methods and apparatus for treating a target site within a blood vessel or other body lumen using two or more catheters or other devices introduced to the target site from opposite sides of the body lumen. Preferably, such improved treatment methods and apparatus could rely on formation of additional access penetrations and/or placement of a guidewire according to other aspects of the present invention. At least some of these objectives will be met by the inventions described hereinafter.

2. Description of the Background Art

Guidewires deployed between spaced-apart access sites in a blood vessel or graft are described in U.S. Pat. Nos. 5,824,040 and 5,766,191. Vascular interventional procedures performed using pairs of catheters advanced from opposite directions to a treatment site are described in U.S. Pat. Nos. 4,873,978 and 4,621,636. Catheters and methods for forming lateral penetrations through tissue to and from blood vessels are described in U.S. Pat. Nos. 5,443,497; 5,429,144; 5,409,019; 5,287,861; WO 97/13463; and WO 97/13471. See also U.S. Pat. No. 5,899,909, which describes a curved instrument for passage into the vagina and outward through the abdomen.

### SUMMARY OF THE INVENTION

According to the present invention, improved methods and apparatus are provided for accessing target sites in a body lumen, particularly natural and artificial blood vessels, such as arteries, veins, autologous grafts, synthetic grafts (i.e., formed from synthetic materials and constructs), arterio-venous fistulas, and the like, which may be located in the peripheral, coronary, or cerebral vasculature. The target sites may be located within a fairly small region, typically having a length of several millimeters or less, or may extend over a relatively long region having a length of 10 cm or greater, often 50 cm or greater, and sometimes as much as 300 cm or greater. In the case of diseased target regions, the disease legions may be continuous or may be segmented throughout the target region within the body lumen where it is desired to perform a diagnostic and/or an interventional procedure. Exemplary diagnostic procedures include imaging, such as ultrasonic imaging, fiberoptic imaging, optical coherence tomography (OCT) imaging, etc.; angiography; flow measurement, such as hot-wire flow measurement, Döppler flow measurement, etc.; diametrical measurement, mechanical measurement, intravascular ultrasound (IVUS), and the like. Exemplary interventional techniques will usually involve the removal or other treatment of a diseased region within the body lumen, such as treatment of occluded regions within the vasculature. Exemplary vascular interventional techniques include angioplasty, atherectomy, embolectomy, luminal prosthesis, (stent or graft) placement, radiation treatment, filtration, aspiration and/or infusion, thrombectomy, thrombolysis, lysis, endarterectomy, anastomic access, surgical bypass, vessel harvesting, and the like.

In particular, the present invention provides improved techniques for accessing a luminal target site from at least two sides, e.g., the upstream side and the downstream side from a target site in the vasculature. Such two-sided access permits multiple and/or sequential treatments to be formed at the target site from both sides. For example, a catheter could be placed on one side of the treatment site to provide for occlusion and/or aspiration of the target site. A catheter placed on the opposite side of the target site could then be used to dislodge stenotic material and direct that dislodged material into the downstream catheter. A second example would be to use first and second catheters having balloons or other occlusive elements at their distal ends. The two catheters could be used to isolate a treatment region within the blood vessel permitting the introduction of thrombolytic agents, embolectomy devices, or the like, with a greatly reduced risk of emboli release. These two techniques are offered as examples only, and a wide variety of other combination treatments will be enabled using the access and treatment methods and apparatus of the present invention.

In a first specific aspect of the present invention, at least a second access penetration is formed in a wall of a body lumen having a first access penetration in the wall. The first access penetration may be formed by any conventional technique, such as a surgical cut-down procedure, the Seldinger or other percutaneous access technique, or the like. A penetrating device is introduced inwardly through the first access penetration into the body lumen. Optionally, the first access penetration may be formed as part of the method of the present invention using the devices of the present invention, as discussed in more detail below. In either case a penetrating element on a penetrating device is then positioned at a target site on the inner surface of the wall, and the penetrating element is advanced outwardly through the wall and overlying tissue to form the second access penetration. The second access penetration will pass through all tissue overlying the body lumen so that the resulting second access penetration opens to a region which permits access back into the body lumen. In the case of blood vessels, both the first access penetration and the second access penetration may both be percutaneous, i.e., from the surface of the patient's skin into the blood vessel being accessed. In the case of certain internal organs, such as the fallopian tubes, access may be to and from a space overlying the organ, e.g., an insufflated region over the organ in a minimally invasive procedure.

Usually, introducing the penetrating device comprises introducing a catheter having a lumen therethrough to the target site. The penetrating device is then pushed from the catheter, causing the penetrating element to deflect laterally as it advances from the catheter. Thus, the penetrating element will be able to penetrate and pass through the luminal wall as it is advanced. In order to assure that the penetrating element proceeds in the proper direction, the penetrating device will usually be rotated to properly align the penetrating element prior to advancement and tissue penetration. For example, markers or other indicia may be provided on the penetrating device and/or the catheter in order to assure the proper rotational alignment of the penetrating element prior to advancement. Alternatively or additionally, the penetrating device may be imaged, e.g., fluoroscopically, prior to and during advancement in order to assure that it is oriented properly. Further optionally, a stabilizing device or mechanism may be provided on the catheter to facilitate pushing the penetrating device from the catheter into the tissue overlying the lumen. Catheters which extend a long distance from the first access penetration and/or which extend through highly tortuous regions of the vasculature or other body lumens present particular difficulties in deploying the penetrating element. The penetrating element will usually comprise an elongate stylet or other sharpened penetrating element (as described in more detail below) which is advanced through tissue by pushing at its proximal end. Pushing such an elongate element through a long and/or twisted catheter can be very difficult since the pushing will often deform the catheter and/or body lumen, reducing the penetrating force achieved at the tip of the device. Moreover, deformation of the catheter and/or lumen can also displace the distal end of the catheter, making precise location of the second access penetration problematic.

To overcome these difficulties, it is desirable to stabilize the catheter within the body lumen prior to and during advancement of the penetrating element through the overlying tissue. This can be achieved in various ways. Most simply, an anchor such as a relatively short balloon can be provided near the distal end of the catheter to hold the catheter in place (at least at its distal end) while the penetrating element is advanced. While an improvement, anchoring the catheter only at its distal end does little to reduce deformation of the catheter and/or body lumen over its proximal regions. Thus, in some cases, it may desirable to anchor the catheter over a major portion of its length, and optionally its entire length, e.g., using a balloon which extends over the entire length of the catheter. As a further alternative, the catheter may be selectively stiffened after it is deployed. Stiffening could be provided by inflating the walls of the catheter, actuating a mechanical linkage within the catheter which locks the catheter shape in place, and the like. As a further alternative, the catheter could be formed at least partly from a highly flexible structure, such as a coil spring, with one or more axial wires or other tethers which prevent elongation of the structure as the penetrating element is passed therethrough. As a still further alternative, co-axial support tubes and other structures could also be provided for selectively stiffening the catheter to enhance pushability of the penetrating device therethrough.

The penetrating device preferably comprises a guide tube having a lumen therethrough where the penetrating element is removably disposed within the lumen of the guide tube. In this way, after the penetrating device has been advanced through the luminal wall to form the second access penetration, the penetrating element can be withdrawn to leave the lumen in the guide tube available to define a guide path between the second access penetration and the first access penetration. In particular, the lumen in the guide tube will be useful for positioning a guidewire, catheter, filament, wire, optical fiber, or other flexible elongate member between the two access penetrations. After the guidewire or other member is placed through the guide tube, the guide tube can be withdrawn. Optionally, a conventional access sheath may then be placed over the guidewire so that catheters and other conventional tools may then be introduced into the vascular or other body lumen through the introducer sheaths on both sides of the target site. More importantly, a single guidewire is now in place for introduction of the interventional catheters and other tools from both sides of the target site. The single guidewire may be appropriately tensioned to facilitate introduction, e.g., using a clamp or other tool or by applying manual tension. Moreover, the elimination of a second guidewire near the target site greatly simplifies deployment and use of all diagnostic and interventional tools.

In a second aspect of the present invention, methods for positioning a guidewire between a first access penetration and a second access penetration comprise positioning a guide tube therebetween. A guidewire is passed through the guide tube, and the guide tube then removed leaving the guidewire in place. Placement of the guide tube is preferably, but not necessarily, effected using the penetrating devices and methods described above. Once in place, the guidewire and optionally placed introducer sheaths can be used for performing the different diagnostic and interventional techniques described elsewhere in this application.

In a third aspect, methods according to the present invention provide for intervention at a target site in a body lumen. A guidewire is positioned between a first access penetration and a second access penetration within the body lumen. A first catheter is then introduced through the first access location over the guidewire to one side of the target site. A second catheter is then introduced through the second access location over the guidewire to the other side of the target site. An intervention is then performed at the target site using at least one of the catheters, preferably using both catheters. The intervention may be diagnostic or therapeutic, as described above, and the body lumen is preferably a blood vessel, more preferably a blood vessel selected from the group listed above. Exemplary interventional techniques comprise deploying occluding elements from at least one of the catheters, preferably from both of the catheters. Such interventional techniques may further comprise disrupting material from within the body lumen and collecting the disrupted and dislodged materials using either or both of the catheters.

Methods of the present invention can also be used for placing two or more guidewires or other flexible elongate members between multiple access penetrations. For example, a single penetrating device introduced through a first access penetration could be used to form two or more additional penetrations in a common body lumen. Alternatively, two or more penetrating devices could be used to form multiple pairs of overlapping or non-overlapping access penetrations. Guidewires or other flexible elongate members could then be passed through the multiple access penetrations in various patterns and combinations.

Devices according to the present invention for positioning a guidewire in a body lumen comprise a catheter which can be introduced through a first access penetration into the body lumen. A means advancable from the catheter for creating a second access penetration provides a guidewire path between the first and second access penetrations. The catheter may have a generally tubular construction with at least one lumen therethrough. The advancable means is preferably reciprocatably received in the catheter lumen and may be extended from a distal luminal opening in the catheter. Preferably, the advancable means has a pre-formed tip which deflects laterally as it is advanced from the catheter. For example, the tip may be formed at least in part from a resilient or shape memory metal, such as stainless steel, Elgiloy™, or Nitinol™. When constrained in the 5 catheter, the tip will remain generally straight. When advanced forwardly from the catheter, the tip will deflect according to its pre-formed memory. Usually, the tip will be sharpened or otherwise shaped so that it will penetrate through the luminal wall as it is advanced (and optionally rotated) through the catheter.

In an exemplary embodiment, the advancable means comprises a guide tube having a penetrating element in a lumen thereof. The penetrating element may be a metal e.g., stainless steel, Elgiloy™, or Nitinol™, or plastic stylet having a sharpened distal tip where the tip extends forwardly of the guide tube. Alternatively, the advancable means can be an integral structure having a sharpened distal tip. The use of a separate guide tube is advantageous, however, since withdrawal of the penetrating element leaves a lumen accessible for placement of a guidewire after the second access penetration has been formed. Optionally, the catheter may comprise a balloon or other expandable anchor disposed at or near its distal end. Further optionally, the device may comprise an support tube for placement over or through the catheter. The support tube is advantageous in that it provides additional column support to the catheter to facilitate pushing of the advancable means from the catheter through the luminal wall.

The present invention still further comprises kits including a penetrating device having a penetrating element in combination with instruction for use according to the methods of the present invention as set forth above. Alternatively, kits could comprise a guide tube in combination with instructions for use setting forth the methods of the present invention as described above. The kits will usually further comprise packaging for holding the kit components together. Conventional packages include boxes, trays, tubes, bags, and the like. Usually, at least some of the kit components will be maintained sterilely within the packaging. The instructions for use may be printed on a separate sheet of paper or may be printed in whole or in part on the packaging itself.

### BRIEF DESCRIPTION OF THE DRAWINGS

FIG. 1 illustrates a penetrating device comprising a catheter, a guide tube, and a stylet constructed in accordance with the principles of the present invention.

FIGS. 2A-2D illustrate functioning of the penetrating device of FIG. 1 and placement of an elongate flexible filament therethrough.

FIGS. 3A-3F illustrate use of the penetrating device of FIG. 1 for forming a second access penetration and placing a guidewire between a first access penetration and the second access penetration.

FIG. 4 illustrates use of the guidewire as placed by the procedure of FIGS. 3A-3F for introducing a single interventional device through the first access penetration.

FIGS. 5A-5B illustrate the introduction of a pair of interventional devices over a guidewire placed by the procedure of FIGS. 3A-3F.

FIG. 6 illustrates employment of a pair of penetrating devices for placing a pair of overlapping guidewires in a single blood vessel.

FIG. 7 illustrates use of a pair of penetrating devices for placing a pair of guidewires or other filaments in a branching blood vessel.

FIGS. 8A and 8B illustrate use of a balloon anchor at the distal end of the penetrating device of the present invention for facilitating forming a second access penetration.

FIGS. 9A and 9B illustrate an elongate anchoring balloon placed over substantially the entire length for anchoring the device and facilitating formation of the second access penetration.

FIG. 10A illustrates a kit constructed in accordance with the principles of the present invention.

### DESCRIPTION OF THE SPECIFIC EMBODIMENTS

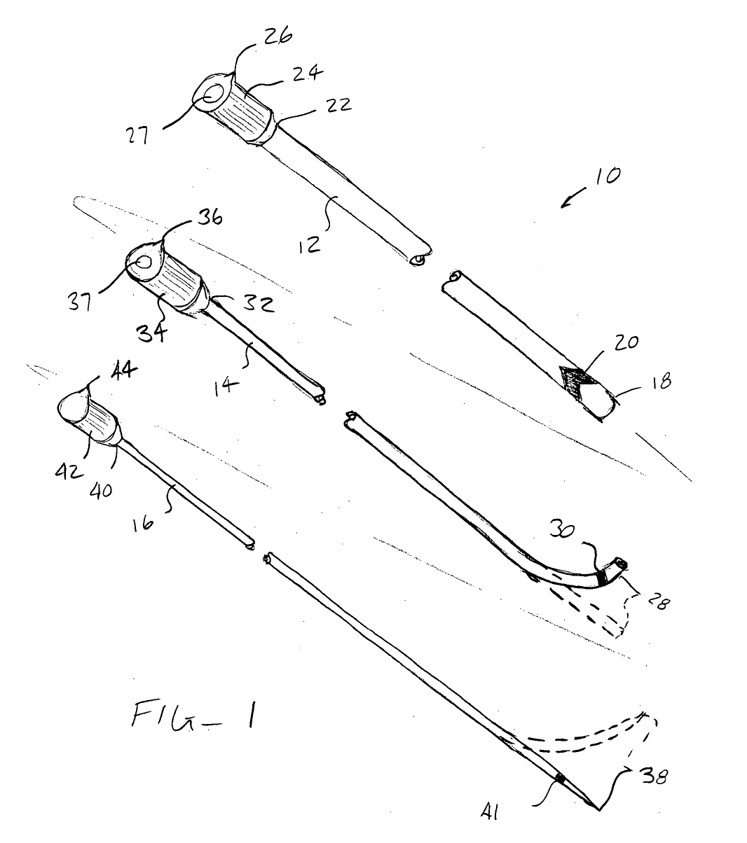

An exemplary penetrating device 10 constructed in accordance with the principles of the present invention is illustrated in FIG. 1. The penetrating device 10 comprises a catheter 12, a guide tube 14, and an elongate penetrating element 16, typically in the form of a sharpened stylet. The catheter 12 has a distal end 18 with a radiopaque marker 20 spaced a short distance therefrom. The catheter has a proximal end 22 with a hub 24 having a pointer 26 formed thereon. The pointer 26 and radiopaque marker 20 are configured so that the marker 20 will have a particular, unique pattern when viewed under fluoroscopic imaging when the pointer is at a pre-defined rotational orientation, typically pointed upwardly. As described in more detail hereinafter, observation of both the radiopaque marker 20 and pointer 26 during a procedure can help orient use of the device 10 to form a second access penetration in a particular direction.

The guide tube 14 also has a distal end 28 with a radiopaque marker 30 spaced a short distance therefrom. The radiopaque marker 30 may also have a unique configuration to permit termination of its rotational orientation under two-dimensional fluoroscopic imaging. The hub 34 is mounted at proximal end 32 of this penetrating member, and a pointer 36 is aligned with the rotational marker 30 in a pre-determined fashion.

The guide tube 14 is received within a lumen 27 of the catheter 12. The guide tube also has a deflectable tip so that it can transition between a straight configuration (shown in broken line) and a curved configuration shown in full line. Optionally, the deflectable tip may be pre-formed so that it lies in its curved configuration absent constraint or external forces. Alternatively, the tip could be pre-formed to lie in the straight configuration. As discussed below, the distal end 38 of the elongate penetrating element 16 may also have a pre-formed curve or straight configuration. The shapes of the distal ends of both the guide tube 14 and penetrating element 16 will be selected so that together they will have a deflected bias, with either or both of the structures contributing to the deflection.

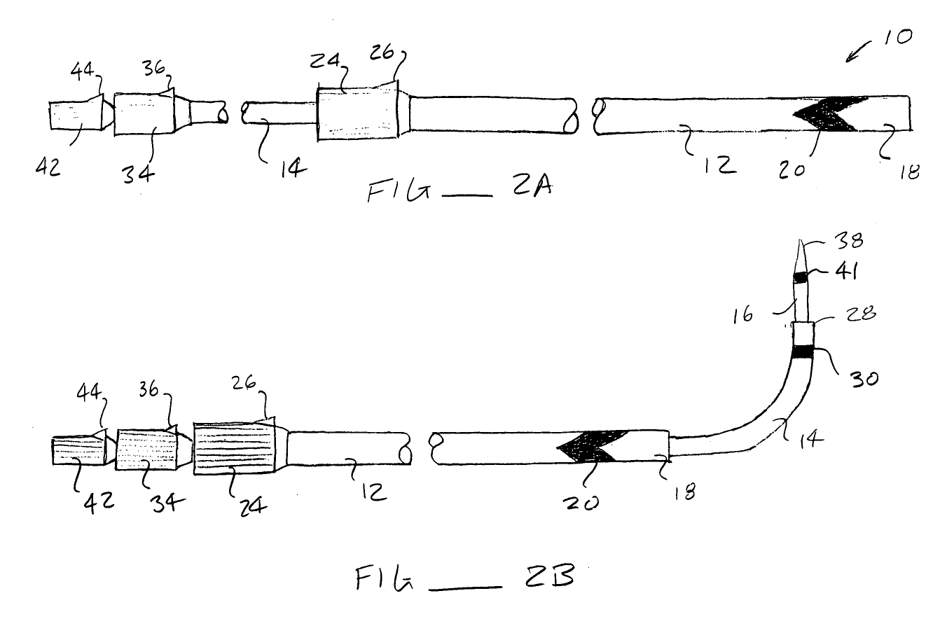

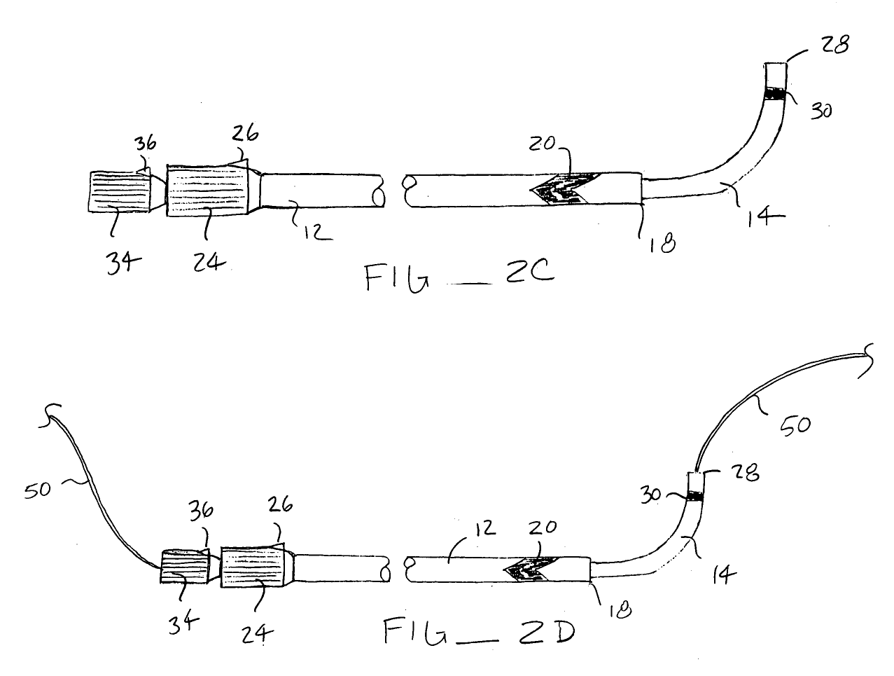

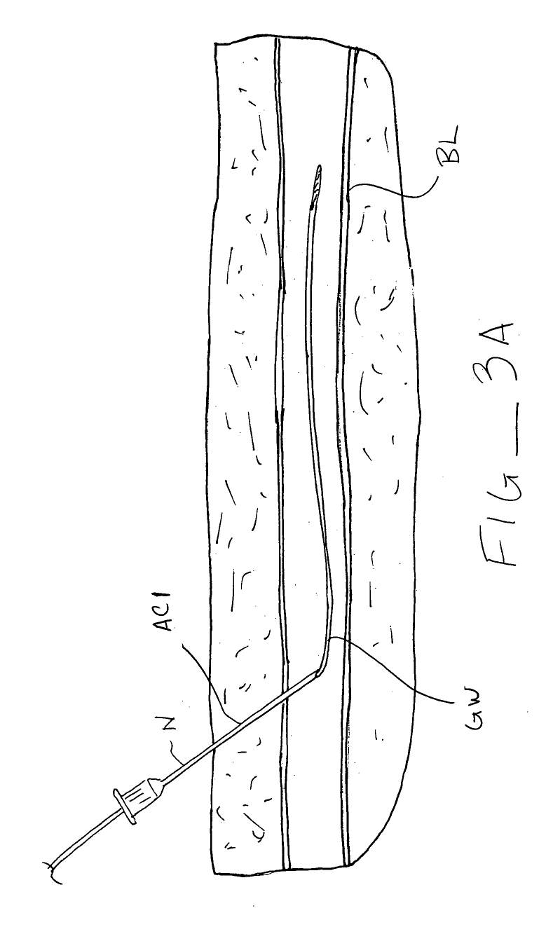

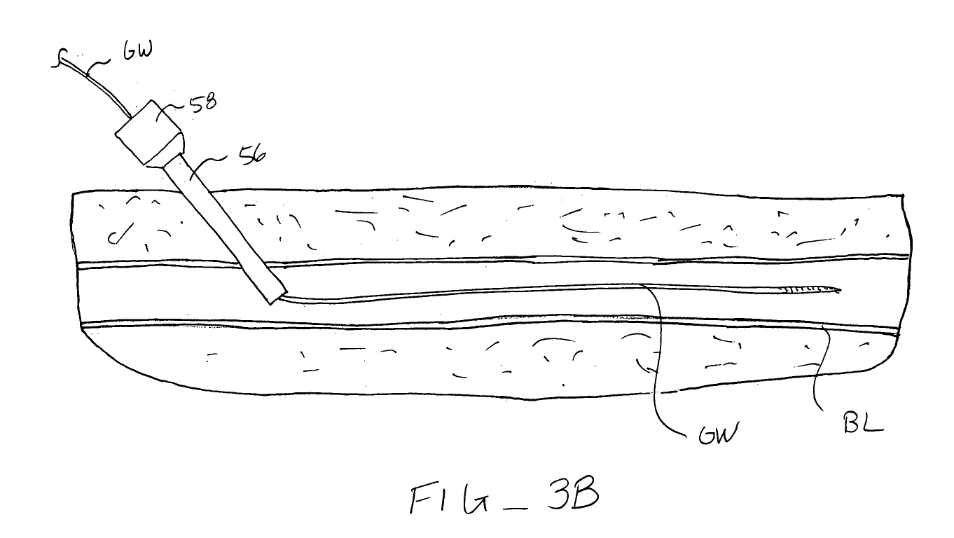

The elongate penetrating element (stylet) 16 has a sharpened distal tip 38 which is also deflectable. A hub 42 having a pointer 44 is attached to proximal end 40 of the stylet, and a radiopaque marker 41 located near the distal tip 38. Optionally, the marker 41 may have a geometry which permits rotational orientation to be determined based on the fluoroscope image. The stylet is received within a lumen 37 of the guide tube 14. In this way, catheter 12, guide tube 14, and elongate penetrating element 16 may be mounted co-axially to form an integrated device 10 having three separately moveable components, as illustrated in FIGS. 2A-2D.

Referring now to FIGS. 2A-2D, a sub-assembly of the guide tube 14 and elongate penetrating element 16 may be mounted within the catheter 12 with the combined hubs 42 and 34 drawn proximally a sufficient distance so that the distal ends of the guide tube 14 and penetrating element 16 are drawn within the distal end 18 of the catheter. In this configuration, the distal ends 28 and 38 of the guide tube 14 and elongate penetrating element 16, respectively, are both straightened so that the catheter can be introduced to a body lumen, as described in more detail hereinafter.

Axial advancement of the assembly of the guide tube 14 and penetrating element 16 causes the distal tips 28 and 38, respectively, to emerge from the distal end 18 of the catheter, as illustrated in FIG. 2B. The combined distal ends bend and deflect in a generally upward direction with the pointers 26, 36, and 44, being aligned with each other and pointing in the same upward direction. The radiopaque marker 20 will also be configured so that this rotational orientation can be observed when viewing downwardly. Use of the pointers and radiopaque marker will be a great aid to the physician in assuring that the guide tube 14 and penetrating element 16 emerge from the catheter 12 in the proper direction so that they pass out of the body lumen and through the overlying tissue at the proper location.

The elongate penetrating element 16 may be withdrawn from the sub-assembly of the catheter 12 and guide tube 14, as shown in FIG. 2C. As illustrated, the distal end of the guide tube 14 remains deflected in this particular embodiment. In other embodiments, where the distal end of the penetrating element 16 is responsible for deflection, the distal end of the guide tube 14 would return into a generally straightened configuration in the absence of the penetrating element 16. Of course, in use, the distal end would generally be constrained within a tissue tract which has been formed, thus holding the guide tube 14 in its desired geometry for further use.

After the penetrating element 16 has been withdrawn, a filament 50, typically a guidewire, may be passed through the lumen 37 of the guide tube, thus being directed through the first access penetration and second access penetration, as described in greater detail. By then withdrawing both the guide tube 14 and catheter 12 from over the guidewire or other filament 50, only the filament will remain in place. The filament, of course, will be available for placement of hemostatic sheaths, positioning of diagnostic and interventional devices, and a wide variety of other purposes, also as discussed in more detail below.

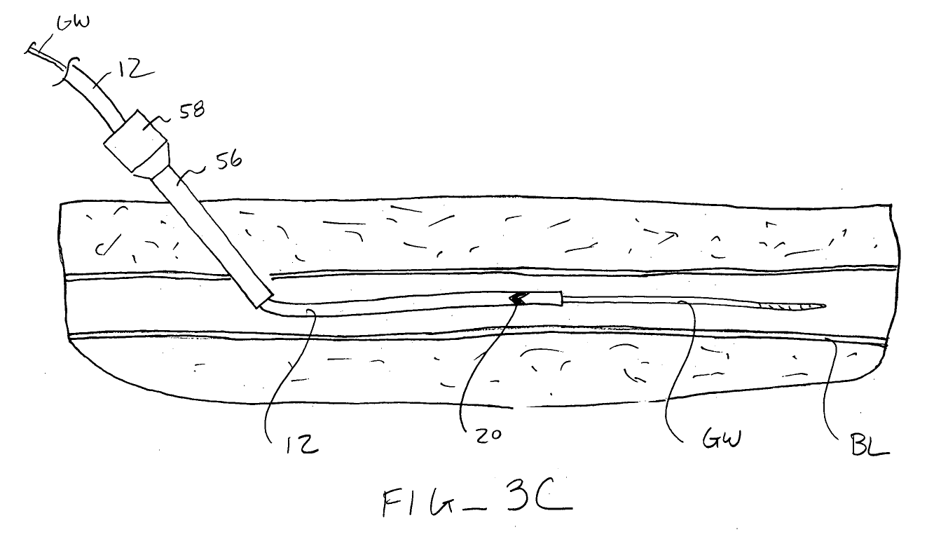

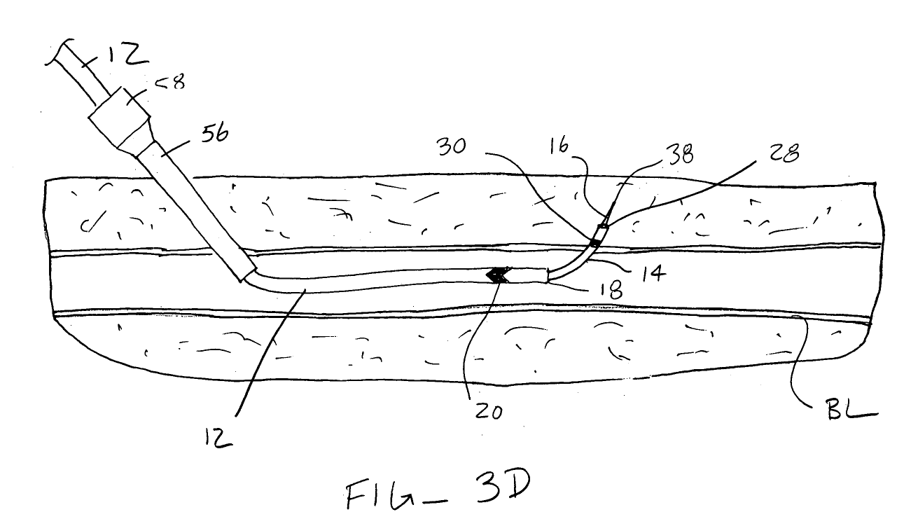

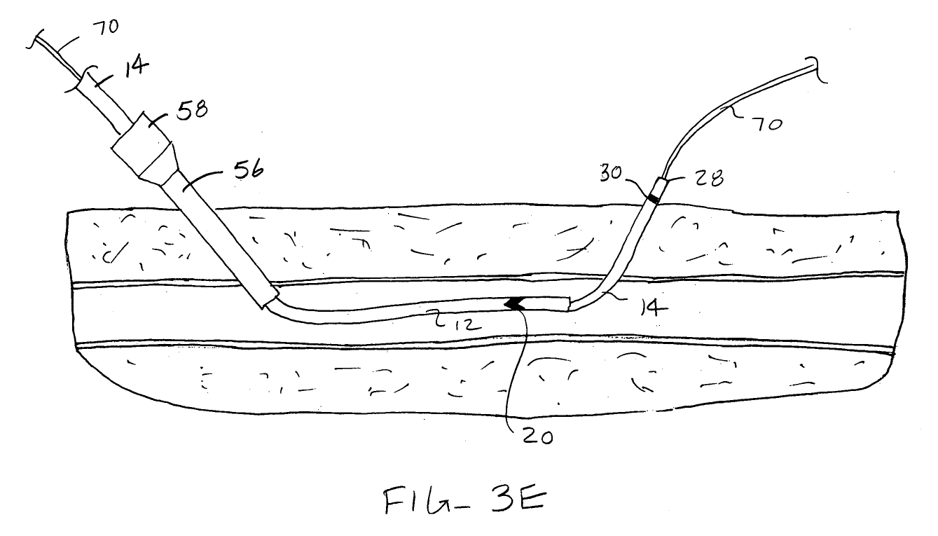

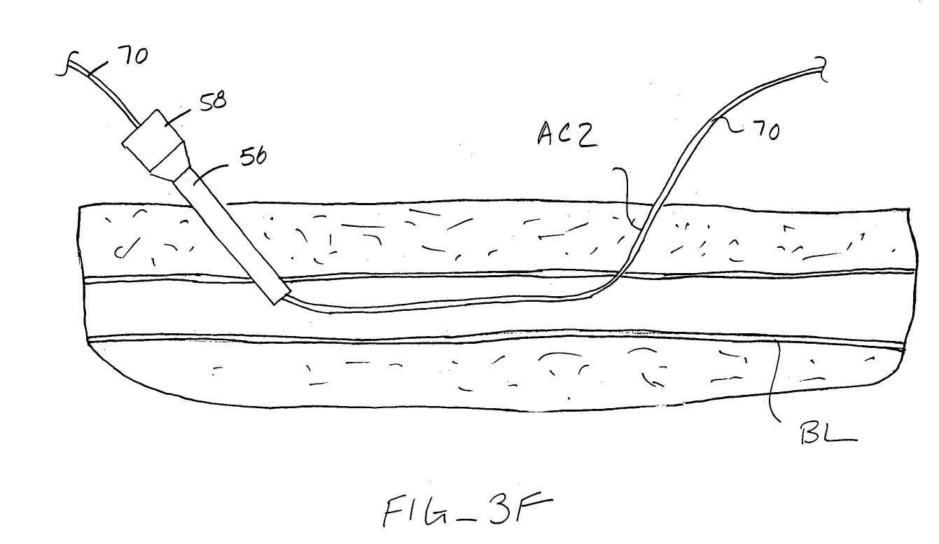

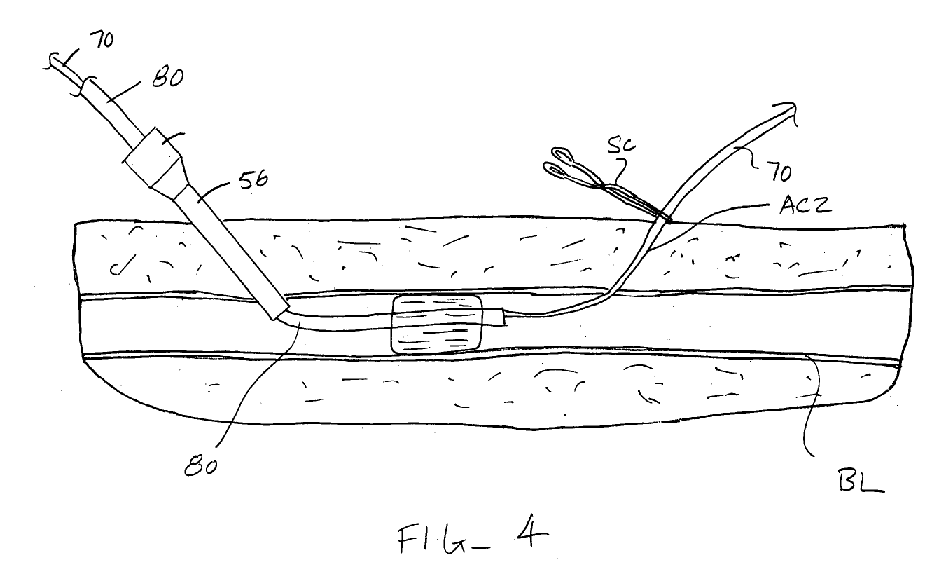

Use of the penetrating device 10 in accessing a body lumen BL, typically a blood vessel of the type described above, will be described with reference to FIGS. 3A-3E. Initially, a conventional or specialized guidewire GW may be introduced to the body lumen BL in a conventional manner, such as using a needle N in a Seldinger technique. The needle thus forms a first access penetration AC1, and after withdrawing the needle, an introducer sheath 56, typically having a hemostatic valve 58, may be introduced over the guidewire GW, as illustrated in FIG. 3B.

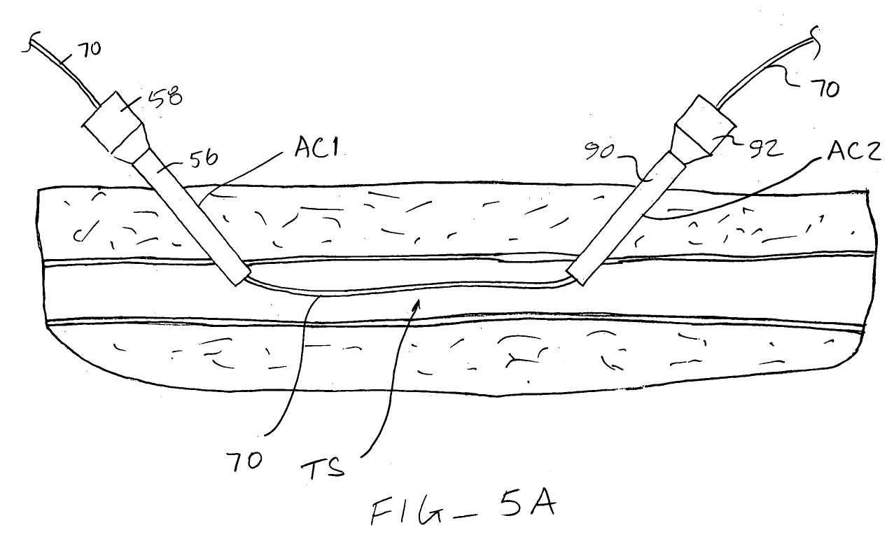

After the introducer sheath 56 has been placed, the penetrating device 10 may be introduced through the sheath 56, either over or in the absence of the guidewire GW. Usually, the guidewire GW will be left in place and the catheter 12 introduced over the guidewire, as shown in FIG. 3C. Once the catheter 12 is located near the target site for the second access penetration (which can be confirmed by fluoroscopic visualization of the marker 20), the guidewire GW may be removed. The assembly of the guide tube 14 and the elongate penetrating member 16 may then be introduced through the catheter 12 so that the distal end extends out through distal tip 18 and deflects so that the sharpened tip 38 penetrates through the luminal wall and into the tissue overlying the lumen, as shown in FIG. 3D. The guide tube is pushed further so that it penetrates through the entire layer of overlying tissue, after which time the elongate penetrating member 16 can be removed. Removal of the penetrating member 16 leaves the lumen 37 available for placement of a second guidewire 70, as shown in FIG. 3E. With the guidewire 70 in place, the guide tube 14 and catheter 12 may be withdrawn, leaving the guidewire 70 in place for a variety of uses, as illustrated in FIG. 3F. A distal portion of the guidewire 70 will pass outwardly through a second access penetration AC2 which has now been formed. The guidewire 70 may be used to introduce a single interventional or diagnostic device, as generally illustrated in FIG. 4. For example, the end of the guidewire which passes out through the second access penetration AC2 may be tensioned, e.g., by clamping with a surgical clamp SC. Alternatively, the surgeon may simply manually tension the guidewire. A device 80, shown in the form of an angioplasty catheter, may then be introduced through the sheath 56 and over the guidewire 70 while said guidewire remains under tension. The ability to tension the guidewire is very advantageous when the device is passed through tortuous regions of a blood vessel or other body lumen and/or through highly occluded regions which are otherwise difficult to pass.

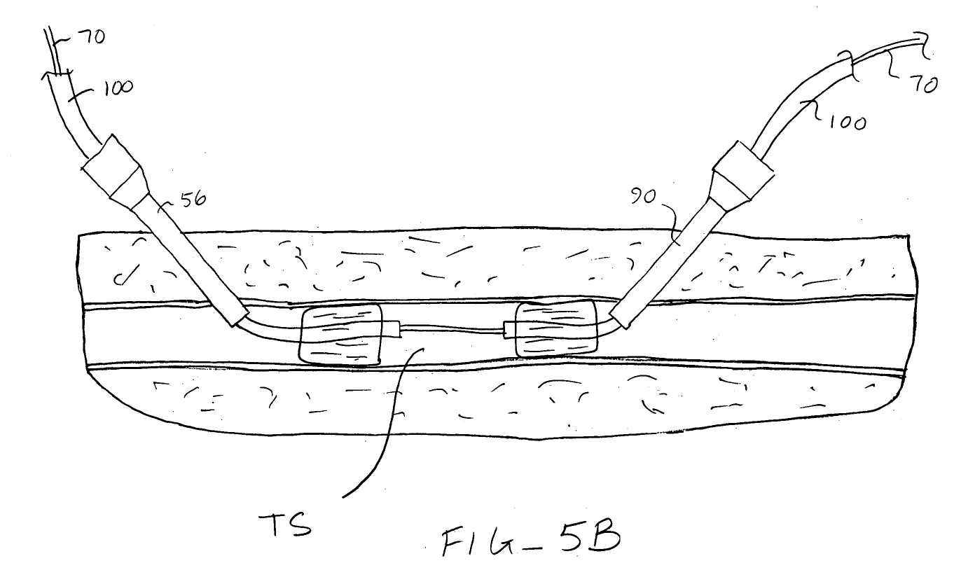

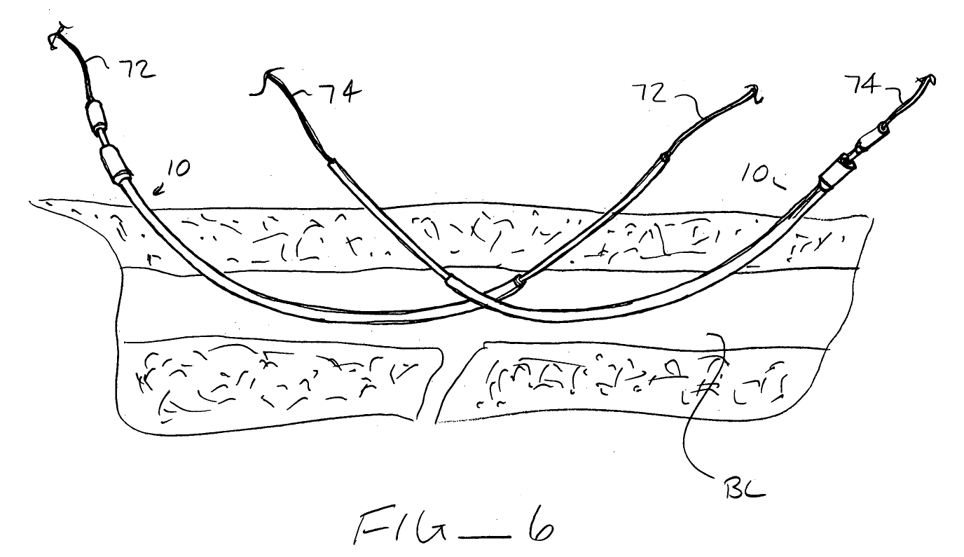

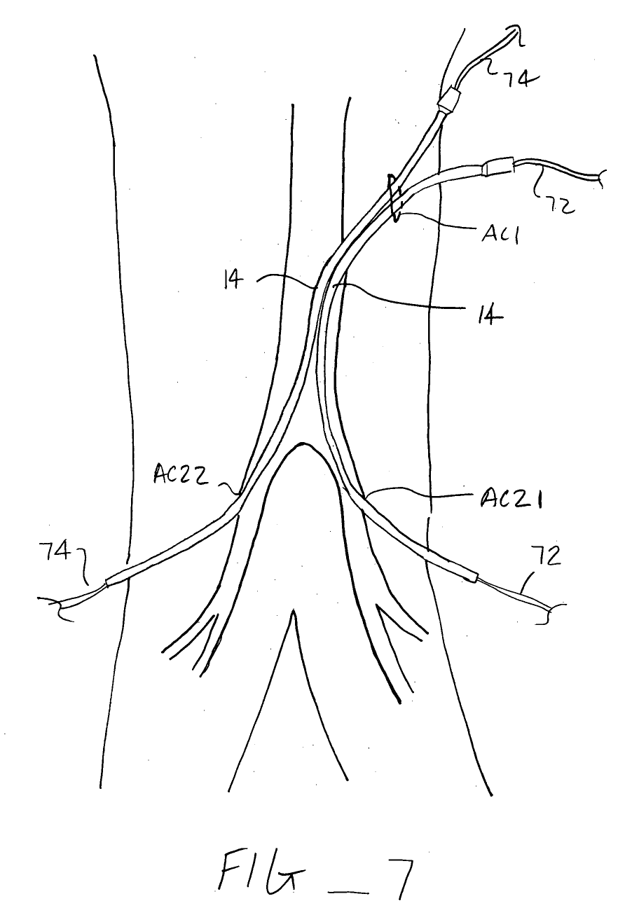

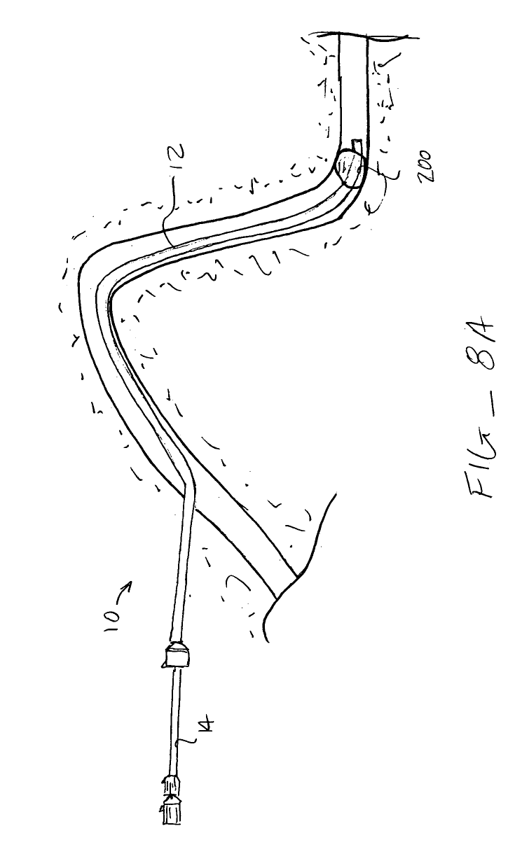

The methods and devices of the present invention are particularly useful for permitting access to target locations from two different sides, as illustrated in FIGS. 5A and 5B. Usually, although not necessarily, a second access sheath 90, usually having a hemostatic hub 92 is placed over the guidewire 70 through the second access penetration AC2. Thus, the guidewire 70 extends between the two sheaths 56 and 90 forming a single rail or track to permit introduction of conventional or other devices toward a target site TS therebetween, as illustrated in FIG. 5A. In one specific embodiment, a pair of occluding catheters 100 can be introduced through the sheaths 56 and 90 over the guidewire 70, as illustrated in FIG. 5B. The balloons, malacots, or other expansible structures may then be expanded to isolate the target site TS therebetween. A variety of procedures can then be performed, such as thrombectomy, thrombolysis, lysis, or the like, with the region remaining isolated and the risk of emboli release and/or blood loss being greatly reduced. In particular, after the invention has been completed, the region can be aspirated to remove any emboli which may have been created. Although illustrated as a pair of isolation catheters, a wide variety of other interventional and diagnostic catheters could be introduced, particularly where the function of one catheter device compliments that of the other. The penetrating devices 10 of the present invention may also be used to deploy two or more catheters, as generally shown in FIG. 6 and 7. In FIG. 6, devices 10 are deployed in a single body lumen with the access penetrations being formed in an overlapping configuration. Thus, the pair of guidewires 72 and 74 can be deployed, also in an overlapping configuration. In FIG. 7, guide tubes 14 are illustrated as deploying guidewires 72 and 74 from a common access penetration AC1 to a pair of secondary access penetrations AC21 and AC22 at a bifurcated region of the vasculature. In particular, the guide tubes 14 are shown entering into the abdominal aorta and exiting through the iliac arteries. It will be appreciated that the penetrating devices 10 of the present invention can be used to deploy two, three, four, or even more guidewires to permit deployment of an even greater number of diagnostic and/or therapeutic devices for performing procedures in the body lumens.

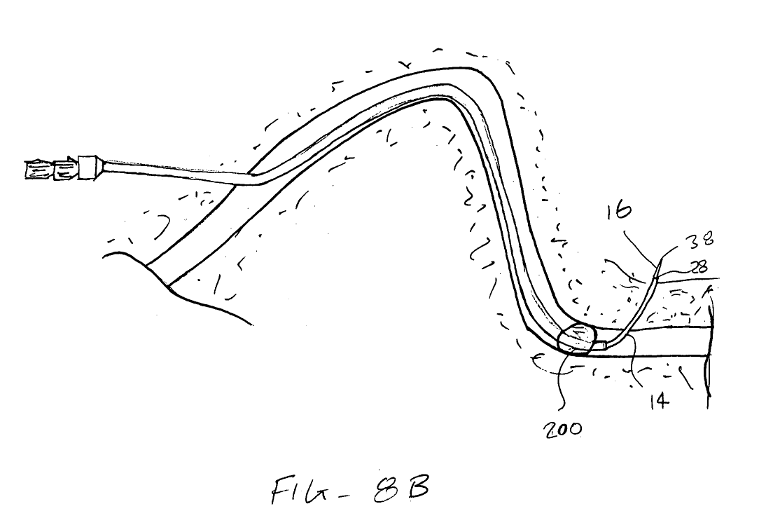

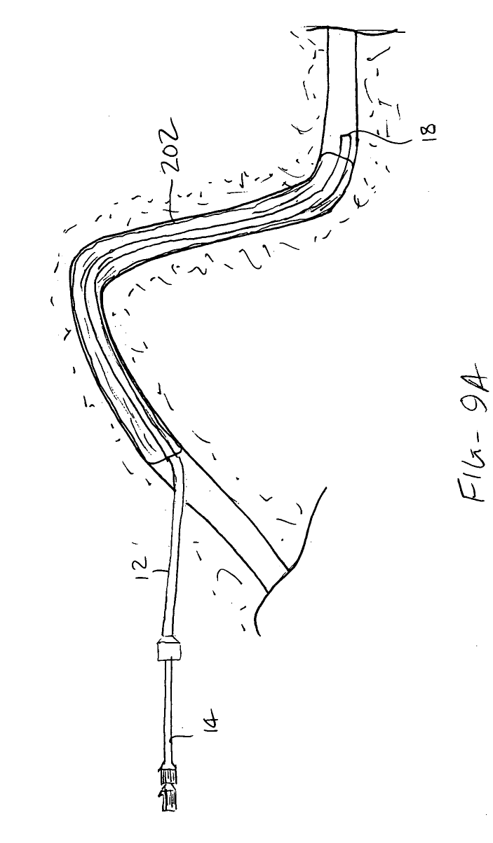

In many cases, it will be desirable to provide for stabilization of the catheter portion of the penetrating device of the present invention. By stabilization, it is meant that at least a portion of the catheter body will be anchored or stiffened within the body lumen in which it is deployed. Most simply, an anchoring balloon, malacot, or other expansible structure 200 can be provided at the distal end of the catheter 12 of a system 10, as illustrated in FIGS. 8A and 8B. By anchoring the distal end of the catheter 12, the distal ends 28 and 38 of the guide tube 14 and penetrating element 16 will be able to push against the anchor rather than pushing against and repositioning the entire catheter body. The anchor 100 will help assure that the device 10 does not move during a formation of the second access penetration.

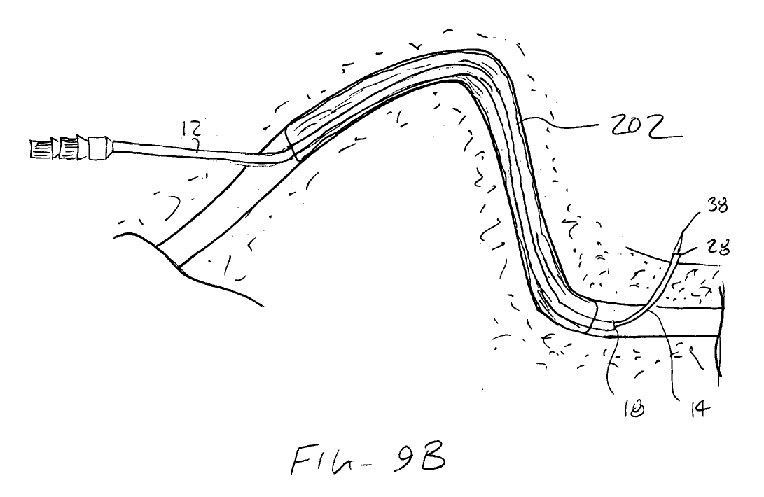

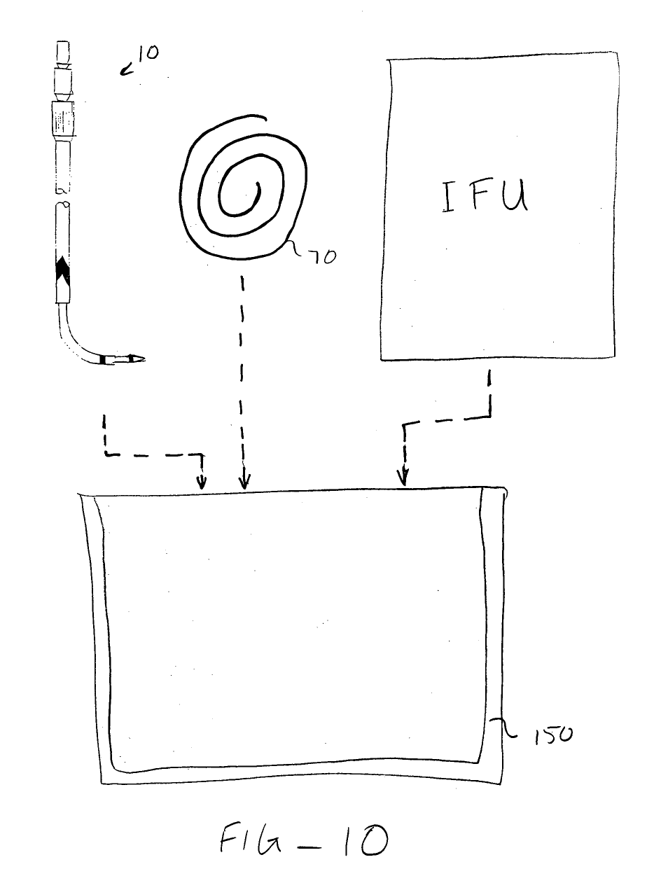

In some instances, use of a single relatively small anchoring device at the distal end of the catheter 12 will be insufficient. In such cases, it will be desirable to provide for an anchor or stiffening element over a long segment of the catheter 12, as illustrated in FIGS. 9A and 9B. For example, an elongate balloon 202 which covers a major portion of the length of the catheter 12 may be provided and inflated in a relatively lengthy segment of the body lumen. This elongate balloon 202 will serve as an anchor over a long portion of the catheter, thus reducing the tendency for the catheter to deform or deform the body lumen as the guide tube and penetrating member 14/16 are advanced therethrough. In addition to anchoring the catheter, the elongate balloon will also serve to stiffen the catheter body over at least a portion of its length. It will be appreciated, however, the wide variety of other mechanical linkages and devices could be provided for stiffening and/or anchoring the catheter within the body lumen prior to deployment of the penetrating element.

Referring now to FIG. 10, kits according to the present invention will comprise at least a penetrating device 10, or a component thereof, together with instructions for use IFU and optionally a package 150. Further optionally, the kit may comprise a guidewire 70 and/or other components which will facilitate practice of the methods of the present invention. The instructions for use IFU may set forth any of the methods described above, and the device 10, or component thereof, and the device will usually be packaged in a sterile fashion within the package 150.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for forming a second access penetration in a wall of a body lumen having a first access penetration in said wall, said method comprising:
introducing a penetrating device inwardly through the first access penetration into the body lumen;
positioning a penetrating element of the penetrating device at a target site in the lumen; and
advancing the penetrating element outwardly through the wall of the lumen and overlying tissue to form the second access penetration.

2. A method as in claim 1, wherein introducing the penetrating device comprises introducing a catheter having a lumen therethrough to the target site and pushing the penetrating device from the catheter, wherein the penetrating element deflects laterally so that it passes through the wall as it is advanced.

3. A method as in claim 2, further comprising rotating the penetrating device to aim the penetrating element prior to pushing the penetrating device from the catheter.

4. A method as in claim 3, further comprising viewing a marker on the catheter and/or penetrating device while the device is being rotated to determine when the penetrating device is properly aimed.

5. A method as in claim 2, further comprising anchoring or stiffening at least a portion of the catheter as the penetrating device is pushed from the catheter.

6. A method as in any of claims 1 to 5, wherein the penetrating device comprises a guide tube having a lumen therethrough and the penetrating device within the lumen, further comprising removing the penetrating element from the guide tube after the second access penetration has been formed, whereby the guide tube lumen provides a path between the first access penetration and the second access penetration.

7. A method as in claim 6, further comprising passing a guidewire through the lumen of the guide tube and withdrawing the guide tube to leave the guidewire in place.

8. A method as in any of claims 1 to 5, wherein the body lumen is a blood vessel.

9. A method as in claim 8, wherein the blood vessel is selected from the group consisting of arteries, veins, autologous grafts, artificial grafts, and arterio-venous fistulas.

10. A method for positioning a guidewire in a body lumen, said method comprising:
positioning a guide tube between a first access penetration and a second access penetration into the body lumen;
passing a guidewire through the guide tube, and
withdrawing the guide tube to leave the guidewire in place.

11. A method as in claim 10, wherein the body lumen is a blood vessel.

12. A method as in claim 11, wherein the blood vessel is selected from the group consisting of arteries, veins, autologous grafts, artificial grafts, and arterio-venous fistulas.

13. A method as in any of claims 10 to 12, wherein positioning the guide tube comprises introducing a penetrating device comprising the guide tube and a penetrating element through the first access penetration, positioning the penetrating element of the penetrating device at a target site, advancing the penetrating element outwardly through the wall to form the second access penetration and position the guide tube therein, and withdraw the penetrating element from the guide tube to leave a lumen for receiving the guidewire.

14. A method as in claim 13, wherein positioning the guide tube further comprises introducing a catheter having a lumen therethrough to the target site and pushing the penetrating device from the catheter, wherein the penetrating element deflects laterally through the wall as it is advanced.

15. A method as in claim 14, further comprising rotating the penetrating device to aim the penetrating element prior to pushing the penetrating device from the catheter.

16. A method as in claim 15, further comprising anchoring or stiffening at least a portion of the catheter as the penetrating device is pushed from the catheter.

17. A method as in any of claims 10 to 12, further comprising introducing at least one device over the guidewire through one of the first and second access penetrations after the guide tube has been withdrawn.

18. A method as in claim 17, wherein a second device is introduced over the guidewire simultaneously through the other of the access penetrations.

19. A method for intervening at a target site in a body lumen, said method comprising:
positioning a guidewire between a first access penetration and a second access penetration into the body lumen;
introducing a first device through the first access location over the guidewire to the target site;
introducing a second device through the second access location over the guidewire to the target site; and
intervening at the target site using at least one of the devices.

20. A method as in claim 19, wherein the body lumen is a blood vessel.

21. A method as in claim 20, wherein the blood vessel is selected from the group consisting of arteries, veins, autologous grafts, artificial grafts, and arterio-venous fistulas.

22. A method as in any of claims 19 to 21, wherein intervening comprises using both devices.

23. A method as in claim 22, wherein intervening comprises imaging with at least one of the devices.

24. A method as in claim 22, wherein intervening comprises deploying an occluding element from at least one of the devices.

25. A method as in claim 24, wherein intervening comprises deploying an occluding element from both of the devices to define an isolated region therebetween.

26. A method as in claim 22, wherein intervening comprises disrupting material within the body lumen with one device and collecting the dislodged material with the other device.

27. A method as in claim 19, wherein intervening at the target site comprises using at least one device to perform angioplasty, atherectomy, aspiration, filtering, infusion, mechanical thrombectomy, endarterectomy, luminal prosthesis placement, lysis, or thrombolysis.

28. A method as in claim 19, wherein positioning the guidewire comprises:
positioning a guide tube between the first access penetration and the second access penetration into the body lumen;
passing the guidewire through the guide tube; and
removing the guide tube to leave the guidewire in place.

29. A method as in claim 28, wherein positioning the guide tube comprises introducing a penetrating device comprising the guide tube and a penetrating element through the first access penetration, positioning the penetrating element of the penetrating device at a target site, advancing the penetrating element outwardly through the wall to form the second access penetration and position the guide tube therein, and withdraw the penetrating element from the guide tube to leave a lumen for receiving the guidewire.

30. A method as in claim 29, wherein positioning the guide tube further comprises introducing a catheter having a lumen therethrough to the target site and pushing the penetrating device from the catheter, wherein the penetrating element deflects laterally through the wall as it is advanced.

31. A method as in claim 30, further comprising rotating the penetrating device to aim the penetrating element prior to pushing the penetrating device from the catheter.

32. A method as in claim 30, further comprising anchoring a distal end of the catheter as the penetrating device is pushed from the catheter.

33. A device for positioning a filament in a body lumen, said device comprising:
a catheter which can be introduced through a first access penetration into the body lumen; and
means advancable from the catheter for creating a second access penetration and providing a filament path between said first and second access penetrations.

34. A device as in claim 33, wherein the catheter has at least one lumen therethrough and the advancable means is reciprocatably received in the catheter lumen.

35. A device as in claim 34, wherein the advancable means has a pre-formed tip which deflects laterally as it is advanced from the catheter.

36. A device as in any of claims 33 to 35, wherein the advancable means comprises a guide tube having a lumen therethrough and a penetrating element removable received in the lumen and extending from a distal tip of the guide tube, wherein the penetrating means can be withdrawn from the guide tube after the guide tube has been placed between the access penetrations to leave the guide tube lumen as the filament path.

37. A device as in claim 36, wherein the penetrating element is a stylet.

38. A device as in any of claims 33 to 35, further comprising an expandable anchor disposed over at least a portion of the catheter.

39. A device as in claim 36, further comprising a support tube having a lumen for receiving the guide tube therethrough.

40. A kit comprising:
a penetrating device having a penetrating element, and
instructions for use according to any of claims 1 to 5.

41. A kit comprising:
a guide tube; and
instructions for use according to any of claims 10 to 12.
